(19) United States
(12) Patent Application Publication
(10) Pub. No.: US 2025/0221431 A1
(43) Pub. Date: Jul. 10, 2025

(54) METHOD FOR FEEDING BLACK SOLDIER FLY LARVAE WITH SELENIUM-RICH BACTERIA SOLUTION

(71) Applicant: BIOFORTE BIOTECHNOLOGY (SHENZHEN) CO., LTD., Guangdong (CN)

(72) Inventors: Cimei CAI, Guangdong (CN); Wenfeng HU, Guangdong (CN); Xueling LI, Guangdong (CN); Jianfeng ZHU, Guangdong (CN); Bin HU, Guangdong (CN); Haolin XIAN, Guangdong (CN)

(73) Assignee: BIOFORTE BIOTECHNOLOGY (SHENZHEN) CO., LTD., Guangdong (CN)

(21) Appl. No.: 18/820,239

(22) Filed: Aug. 29, 2024

(30) Foreign Application Priority Data
Jan. 7, 2024 (CN) 202410020118.4

(51) Int. Cl. A23K 20/20; A23K 10/18; A23K 50/90; C12N 1/20

(52) U.S. Cl. CPC A23K 20/30; A23K 10/18; A23K 50/90; C12N 1/20; A23V 2400/113; A23V 2400/157; A23V 2400/169

(57) ABSTRACT

The present invention discloses a method for feeding black soldier fly larvae with a selenium-rich bacteria solution, as follows: inoculating an intestinal bacteria seed liquid of black soldier fly to a fermentation medium at an inoculum size of 1%-3%, and cultivating at 30° C.-37° C. till the log phase to obtain a fermentation broth of log phase; adding sodium selenite to the fermentation broth of log phase to enable the fermentation broth to have a sodium selenite concentration of 10-600 μg/mL, then cultivating the fermentation broth at 30° C.-37° C. for 24-36 h to obtain a selenium-rich bacteria solution; and selecting 4-to-6-day-old black soldier fly larvae to be fed, adding the selenium-rich bacteria solution to a feed to enable the feed to have a selenium content of 5-500 mg/kg (wet weight), feeding the black soldier fly larvae with the feed till the end of the fifth instar of the larvae.

### CROSS-REFERENCE TO RELATED APPLICATION

This application claims the priority benefit of China application no. 202410020118.4, filed on Jan. 7, 2024. The entirety of the above-mentioned patent application is hereby incorporated by reference herein and made a part of this specification.

### TECHNICAL FIELD

The present application relates to the biotechnology field, and specifically, relates to a method for feeding black soldier fly larvae with a selenium-rich bacteria solution, and a product, a preparation method and use thereof.

### BACKGROUND

As a microelement needed for health maintenance of humans and animals, selenium (Se) is honored as “natural antidote” and “King of anti-cancer”. Selenium has physiological functions such as resisting oxidation, anticancer and enhancing immunity, for it is an active center of antioxidant enzymes such as glutathione peroxidase (GPX). Selenium deficiency may cause several diseases including Keshan disease and Kaschin-Beck disease (Marco Vinceti, Filippini Tommaso, Wise Lauren-A. Environmental Selenium and Human Health: an Update [J]. Current Environmental Health Reports, 2018, 5 (4): 464-485). According to the statistics, residents in many countries around the world, including China, are in a state of selenium deficiency, and their daily dietary intake of selenium is much lower than international standards (Wang Liping, Tang Dejian, Shen Yamei, et al. The Status Quo of Nutrient Deficiency and Supplementation Methods of Selenium [J]. Food Industry, 2020, 41 (01): 339-343).

There are many forms of selenium, including inorganic selenium such as sodium selenite and sodium selenite; organic selenium such as selenium polysaccharides, selenoproteins, and selenoamino acids; and nano-selenium. Compared to inorganic selenium such as sodium selenite, organic selenium such as nano-selenium have higher bioavailability and less toxicity, making organic selenium more suitable for being absorbed and utilized by animals and humans.

Black soldier fly (Hermetia illucens L.) is a resource-based insect that has newly emerged in recent years, with a wide range of feeding habits, fast reproduction and growth rates, and can be industrialized for large-scale breeding and production. Moreover, the larvae of black soldier flies are rich in nutrients such as protein, which has great application value in the development and utilization of protein foods, health products, animal feed, and biodiesel (Huang Wanying, Deng Qiuyan, Lai Xiaoqi. Application of Resource-Oriented Management in Black Soldier Fly Breeding [J]. Breeding and Feed, 2022, 21 (04): 49-51.)

Currently, there is little research on black soldier flies enriched with selenium, and the selenium sources used are mainly from inorganic selenium such as sodium selenite. Shen et al used sodium selenite to feed the black soldier fly larvae and verified that selenium element can be absorbed, utilized and accumulated by the black soldier fly larvae. However, with the increase of sodium selenite addition, the selenium exerts an inhibitory effect on the growth of black soldier flies and causes a decrease in the survival rate of black soldier fly larvae (Shen Gaolin. Study on the Optimization of Feeding Condition and Selenium-rich Technology of Black Soldier Fly [D]. Nanchang University, 2016).

A patent application “Preparation method for selenium-rich insect protein powder” filed on 24 Feb. 2020 by Dalian University of Technology (Publication No. CN111109206A) discloses a method for producing selenium-rich insect protein by using inorganic selenium (sodium selenite) and crops as substrates and black soldier flies as carriers. Through preparing selenium-rich feed, conducting selenium-rich breeding processes, and processing selenium-rich insects, a selenium-rich insect protein powder is ultimately obtained. However, this invention uses inorganic selenium as the selenium source. With the increase of inorganic selenium addition, the toxicity of selenium increases, and the survival rate of black soldier fly larvae decreases, resulting in a decrease in conversion rate and production capacity, which is not conducive to the large-scale production of selenium-rich black soldier flies.

Therefore, there is an urgent need of a more efficient feeding method for black soldier flies, which not only promotes the growth of black solider fly larvae, but also reduces the inhibitory effect of the selenium element on the black solider fly larvae and improves the absorption and utilization of the selenium element.

### SUMMARY

Regarding the above issues, the present invention provides a method for feeding black soldier fly larvae with a selenium-rich bacteria solution, and a product, a preparation method and use thereof.

In order to achieve the above objectives, the following technical solutions are adopted.

A method for feeding black soldier fly larvae with a selenium-rich bacteria solution includes the following steps:
step 1, inoculating an intestinal bacteria seed liquid of black soldier fly to a fermentation medium at an inoculum size of 1%-3%, and cultivating at 30° C.-37° C. till the log phase to obtain a fermentation broth of log phase;
step 2, adding sodium selenite to the fermentation broth of log phase to enable the fermentation broth to have a sodium selenite concentration of 10-600 μg/mL, then cultivating the fermentation broth at 30° C.-37° C. for 24-36 h to obtain a selenium-rich bacteria solution; and
step 3, selecting 4-to-6-day-old black soldier fly larvae to be fed, adding the selenium-rich bacteria solution to a feed to enable the feed to have a selenium content of 5-500 mg/kg, controlling a moisture content of the feed to 60%-75%, feeding the black soldier fly larvae with the feed at a dry feed to larva ratio of 0.5-1.0 g per larva in conditions of 28° C.-30° C. feeding temperature and 60%-70% air humidity level, and terminating feeding at the end of fifth instar of the larvae.

Preferably, the intestinal bacteria seed liquid of black soldier fly in step 1 is prepared by the following method: inoculating an intestinal bacterial strain of black soldier fly to a culture medium after activating the intestinal bacterial strain of black soldier fly, and cultivating at 30° C.-37° C. for 12-18 h to reach a bacteria content of 10⁸-10⁹ CFU/mL to serve as the intestinal bacteria seed liquid of black soldier fly.

Preferably, the cultivating in step 1 lasts for 6-12 h, so that the fermentation broth has a bacteria content of 10⁸-10⁹ CFU/mL.

Preferably, the selenium-rich bacteria solution in step 2 has a bacteria content of 10⁹-10¹⁰ CFU/mL.

Preferably, the selenium-rich bacteria solution in step 2 has a nano-selenium content of 5-350 μg/mL.

Specifically, the nano-selenium, as a speciation of selenium element, is a nano sized elemental selenium that can be obtained by converting an inorganic selenium such as sodium selenite. The nano-selenium has multiple bioactivities such as low toxicity, good biocompatibility, antibacterial property and oxidation resistance.

Preferably, the intestinal bacterial strain of black soldier fly is selected from a group consisting of Lactococcus lactis, Lactobacillus acidophilus, Lactobacillus plantarum and Enterococcus faecalis, and complex microbial inoculants thereof with Bacillus subtilis.

Preferably, the feed in step 3 is chicken feed or plant-based feed, with the plant-based feed being prepared by combining bean pulp, bran, and grass meal according to a weight ratio of 3:5:2.

A product of selenium-rich black soldier fly larvae, including selenium-rich black soldier fly larvae, selenium-rich black soldier fly frozen fresh larvae, selenium-rich black soldier fly dry larvae, and selenium-rich black soldier fly larvae protein powder.

A preparation method for selenium-rich black soldier fly dry larvae, including subjecting the above-mentioned selenium-rich black soldier fly larvae of the fifth instar to starvation treatment for 1 day, and then drying the selenium-rich black soldier fly larvae till the moisture content is less than 8% to obtain the dry larvae.

A preparation method for selenium-rich black soldier fly larvae protein powder, including subjecting above-mentioned selenium-rich black soldier fly larvae of the fifth instar to starvation treatment for 1 day, smashing the selenium-rich black soldier fly larvae after drying, then adding petroleum ether at a solid-liquid ratio of 1:60-1:100 g/mL, and processing at 100° C. for 3-6 h to extract larval oil and to obtain defatted selenium-rich black soldier fly larvae protein powder.

Use of selenium-rich black soldier fly larvae in preparing feed for livestock and poultry.

Compared with the prior art, the present invention has the following advantages and beneficial effects:

The selenium-rich bacteria solution of the present invention is prepared by two-step fermentation, in which cultivating an intestinal bacterial strain of black soldier fly till the log phase to obtain a large amount of intestinal bacteria of black soldier fly with high activity, then sodium selenite is added to the fermentation broth to continue the fermentation, where the intestinal bacteria of black soldier fly convert the inorganic selenium into the nano-selenium to obtain the selenium-rich bacteria solution with high nano-selenium content, and finally the selenium-rich bacteria solution is added to the feed for black soldier fly larvae. By combining the advantages of the intestinal bacteria of black soldier fly and the nano-selenium, the selenium-rich bacteria solution may reduce the toxicity or inhibitory effect of inorganic selenium (for example, sodium selenite) on the black soldier fly larvae, is conducive to the growth of larvae by using the intestinal bacteria of black soldier fly, and may enhance accumulation, absorption and utilization of the selenium element. By combining the advantages of larvae nutrition and selenium element, the obtained selenium-rich black soldier fly larvae may promote the growth and productive performance of livestock and poultry, and solve the problems of high-dose toxicity and food safety when directly applying sodium selenite to the breeding of livestock and poultry.

### DETAILED DESCRIPTION OF EMBODIMENTS

The present invention will be further explained in conjunction with specific implementations. The experimental methods in the following examples, unless otherwise specified, are all conventional methods. The test materials used in the following examples, unless otherwise specified, were purchased from conventional biochemical reagent stores. The strains used can be obtained through public means.

### Example 1

Step 1, strains of Lactococcus lactis, Lactobacillus acidophilus, Lactobacillus plantarum, Enterococcus faecalis, Bacillus subtilis derived from intestinal tract of the black soldier fly were respectively activated, then respectively inoculated to a MRS or LB culture medium, and cultivated at 37° C. for 12 h to obtain seed liquids with a bacteria content of 10⁸-10⁹ CFU/mL, respectively;

step 2, the respective seed liquids were each inoculated to a MRS or LB culture medium at an inoculum size of 2%, and fermented for 6 h; then sodium selenite was added to each fermentation broth to enable the fermentation broth to have a selenium content of 450 μg/mL, and each fermentation broth was then cultivated at 37° C. for 36 h to obtain a selenium-rich bacteria solution with a bacteria content of 10⁹ CFU/mL.

A comparative example was set simultaneously, where after the strain in step 1 was activated, sodium selenite was added to a MRS or LB culture medium while the strain was inoculated to the culture medium, so that a selenium content in the medium was 450 μg/mL, and no sodium selenite was added during step 2. The other process was the same as that in Example 1. Nano-selenium contents in the fermentation broths obtained by different fermentation methods were then observed.

Determination method of nano-selenium in the present invention is as follows:

(1) Drawing a standard curve: drawing a standard curve of nano-selenium of 0-800 μg. Specifically, 0.5 mL selenium solution of each group was mixed and reacted with 0.5 mL hydroxylamine solution for 1 h, then 2 mL of 1 mol/L Na₂S solution was added thereto and mixed slowly and evenly. After 1 h, the solution exhibited a reddish brown color and the absorbance value thereof was tested at 500 nm.

(2) Determination of nano-selenium in the bacteria solution: 10 mL bacteria solution was subjected to centrifugation at 10000 r/min for 10 min to obtain bacteria precipitate. The bacteria precipitate was washed 3 times, then a Na₂S solution was added thereto and reacted with the bacteria precipitate for 1 h with intermittently shaking. After centrifugation, the supernatant was taken to determine its absorbance value at 500 nm.

TABLE 1
Nano-selenium content in the fermentation broth

Nano-selenium content (μg/mL)

| | Comparative | The Present |
|---|---|---|
| Strain | Example | Invention |
| Lactobacillus acidophilus | 66.54 ± 0.89 | 117.52 ± 0.86 |
| Lactococcus lactis | 53.44 ± 2.43 | 104.87 ± 3.85 |
| Lactobacillus plantarum | 111.09 ± 14.49 | 186.65 ± 7.72 |
| Enterococcus faecalis | 131.43 ± 7.93 | 257.52 ± 9.37 |
| Bacillus subtilis | 125.32 ± 3.50 | 206.23 ± 4.38 |

It can be concluded from the above results that the two-step fermentation, which consists of fermenting till the log phase followed by adding sodium selenite, is conducive to the reduction of inorganic selenium such as sodium selenite into nano-selenium, and increases the nano-selenium content in the fermentation broth.

### Example 2

Step 1, Enterococcus faecalis derived from intestinal tract of the black soldier fly was activated, then inoculated to a MRS culture medium, and cultivated at 37° C. for 12 h to obtain a seed liquid with a bacteria content of 10⁸-10⁹ CFU/mL;

Step 2, the seed liquid was inoculated to a MRS culture medium at an inoculum size of 2%, and fermented for 6 h; then sodium selenite was added to the fermentation broth to enable the fermentation broth to have a selenium content of 450 μg/mL, and the fermentation broth was then cultivated at 37° C. for 36 h to obtain a selenium-rich bacteria solution with a bacteria content of 10⁹ CFU/mL; and

Step 3, 4-day-old black soldier fly larvae were selected to be fed; the selenium-rich bacteria solution was added to chicken feed to enable the chicken feed to have a total selenium content of 100, 200, 300 mg/kg (wet weight), respectively; a moisture content of the chicken feed was controlled to 60%, then the black soldier fly larvae were fed with the chicken feed at a ratio of dry chicken feed to larva being 116 g per 150 larvae (the ratio of dry feed to larva was 0.77) in conditions of 28° C.-30° C. feeding temperature and 60%-70% air humidity level, and feeding was terminated at the end of the fifth instar of the larvae.

A group without any additives was used as the blank group (Group 1), groups with the addition of sodium selenite were used as the positive groups (Groups 2, 4, and 6), and groups with the addition of the selenium-rich bacteria solutions were used as the experimental groups (Groups 3, 5 and 7). The effects of the selenium-rich bacteria solution on breeding of the selenium-rich black soldier flies were determined according to the indicators, i.e. growth performance of the larvae (survival rate, larva weight), bioconversion efficiency, and selenium content in the larva. See Table 2.

After the feeding was terminated, the obtained larvae were screened, washed, wiped dry and weighed. The survival rate was calculated according to the number of live larvae. The larvae were subjected to starvation for 1 day, then dried and weighed. The residual feed was also dried and weighed. Thereby, bioconversion efficiency was calculated according to the following: bioconversion efficiency=dry weight of the larvae/(dry weight of the added feed−dry weight of the residual feed)×100%. Selenium content in the larva was determined after smashing the dry larvae according to “GB5009.93-2017 Determination of Selenium in Food”.

TABLE 2
Growth performance of selenium-rich black solider fly larvae of Example 2

| | Selenium | | Larva weight | | Selenium |
|---|---|---|---|---|---|
| | content added | Survival | (mg per | Bioconversion | content in the |
| Group | (mg/kg) | rate (%) | larva) | efficiency (%) | larva (μg/g) |
| 1 | 0.00 | 94.45 ± 0.38 | 212.37 ± 5.44 | 17.24 ± 0.77 | ND |
| 2 | 100.00 | 94.77 ± 0.51 | 212.39 ± 9.63 | 17.40 ± 0.52 | 87.85 ± 6.31 |
| 3 | 100.00 | 94.78 ± 0.77 | 215.14 ± 2.81 | 19.97 ± 0.88 | 121.49 ± 6.37 |
| 4 | 200.00 | 94.22 ± 1.35 | 214.37 ± 7.56 | 17.56 ± 0.58 | 169.31 ± 12.79 |
| 5 | 200.00 | 94.44 ± 0.84 | 216.15 ± 4.42 | 23.47 ± 1.31 | 229.29 ± 9.60 |
| 6 | 300.00 | 91.33 ± 4.27 | 210.57 ± 3.50 | 19.23 ± 0.04 | 234.57 ± 6.17 |
| 7 | 300.00 | 94.89 ± 1.02 | 224.72 ± 5.36 | 27.13 ± 0.59 | 324.96 ± 19.34 |

Note:
ND denotes a value below the detectable limit.

It can be concluded from Table 2 that in the case of sufficient nutrition, selenium tolerance of the black soldier fly larvae is relatively strong. Under the condition of the same selenium content added, compared with sodium selenite (the positive groups), the selenium-rich bacteria solution (the experimental groups) can enhance the bioconversion efficiency, improve absorption of the larvae to selenium and increase the selenium content in the larva.

### Example 3

Step 1, Enterococcus faecalis derived from intestinal tract of the black soldier fly was activated, then inoculated to a MRS culture medium, and cultivated at 37° C. for 12 h to obtain a seed liquid with a bacteria content of 10⁸-10⁹ CFU/mL;

Step 2, the seed liquid was inoculated to a MRS culture medium at an inoculum size of 2%, and fermented for 6 h; then sodium selenite was added to the fermentation broth to enable the fermentation broth to have a selenium content of 450 μg/mL, and the fermentation broth was then cultivated at 37° C. for 36 h to obtain a selenium-rich bacteria solution with a bacteria content of 10⁹ CFU/mL; and

Step 3, 6-day-old black soldier fly larvae were selected to be fed; the selenium-rich bacteria solution was added to a feed to enable the feed to have a total selenium content of 100, 200, 300 mg/kg (wet weight), respectively; where the feed consisted of bean pulp, bran and grass meal at a ratio of 3:5:2, a moisture content of the feed was controlled to 75%; then the black soldier fly larvae were fed with the feed at a ratio of 150 g of dry feed per 150 larvae (the ratio of dry feed to larva was 1.0) in conditions of 28° C.-30° C. feeding temperature and 60%-70% air humidity level, and feeding was terminated at the end of the fifth instar of the larvae.

A group without any additives was used as the blank group (Group 1), groups with the addition of sodium selenite were used as the positive groups (Groups 2, 4, and 6), and groups with the addition of the selenium-rich bacteria solutions were used as the experimental groups (Groups 3, 5 and 7). Effects of the selenium-rich bacteria solution on breeding of the selenium-rich black soldier flies were determined according to the indicators, i.e. growth performance of the larvae (survival rate, larva weight), bioconversion efficiency, and selenium content in the larva. See Table 3. The test method for each indicator was the same as that in Example 2.

TABLE 3
Growth performance of selenium-rich black solider fly larvae of Example 3

| | Selenium | | Larva weight | | Selenium |
|---|---|---|---|---|---|
| | content added | Survival | (mg per | Bioconversion | content in the |
| Group | (mg/kg) | rate (%) | larva) | efficiency (%) | larva (μg/g) |
| 1 | 0.00 | 97.11 ± 1.39 | 209.47 ± 5.10 | 11.87 ± 0.13 | ND |
| 2 | 100.00 | 85.04 ± 8.77 | 179.63 ± 9.99 | 8.97 ± 0.87 | 93.56 ± 5.00 |
| 3 | 100.00 | 95.01 ± 1.72 | 199.14 ± 3.98 | 9.42 ± 0.50 | 124.86 ± 5.01 |
| 4 | 200.00 | 84.51 ± 5.06 | 154.41 ± 10.66 | 6.24 ± 0.43 | 241.53 ± 18.04 |
| 5 | 200.00 | 94.54 ± 1.13 | 173.38 ± 6.13 | 8.26 ± 0.60 | 331.32 ± 32.13 |
| 6 | 300.00 | 82.22 ± 8.77 | 119.53 ± 10.79 | 4.69 ± 0.30 | 451.23 ± 10.42 |
| 7 | 300.00 | 93.78 ± 1.54 | 127.97 ± 1.06 | 7.57 ± 0.21 | 582.22 ± 9.93 |

Note:
ND denotes a value below the detectable limit.

It can be concluded from Table 3 that sodium selenite exerts an inhibitory effect on the black soldier fly larvae. Specifically, the larvae survival rate, weight, and bioconversion efficiency drop with the increase of sodium selenite concentration. On the other hand, the selenium-rich bacteria solution (the experimental groups) can reduce the inhibitory effect, improve absorption of the larvae to selenium and increase the selenium content in the larva.

The feed in the present example was prepared by compounding bean pulp, bran and grass meal, with a poorer nutritional condition than the chicken feed in Example 2. By taking Example 2 into account, it is evident that under a poorer nutritional condition, sodium selenite exerts a stronger inhibitory effect on the black soldier fly larvae, while under a better nutritional condition the black soldier fly larvae have stronger tolerance to the inhibitory effect of sodium selenite, and based on this situation, the selenium-rich bacteria solution can further enhance the larvae's absorption of selenium.

### Example 4

Step 1, Lactobacillus plantarum derived from intestinal tract of the black soldier fly was activated, then inoculated to a MRS culture medium, and cultivated at 37° C. for 12 h to obtain a seed liquid with a bacteria content of 10⁸-10⁹ CFU/mL;

Step 2, the seed liquid was inoculated to a MRS culture medium at an inoculum size of 2%, and fermented for 6 h; then sodium selenite was added to the fermentation broth to enable the fermentation broth to have a selenium content of 450 μg/mL, and the fermentation broth was then cultivated at 37° C. for 36 h to obtain a selenium-rich bacteria solution with a bacteria content of 10⁹ CFU/mL; and

Step 3, 6-day-old black soldier fly larvae were selected to be fed; the selenium-rich bacteria solution was added to a feed to enable the feed to have a total selenium content of 100, 200, 300 mg/kg (wet weight), respectively; where the feed consisted of bean pulp, bran and grass meal at a ratio of 3:5:2, a moisture content of the feed was controlled to 75%; then the black soldier fly larvae were fed with the feed at a ratio of dry feed to larva being 150 g per 150 larvae (the ratio of dry feed to larva was 1.0) in conditions of 28° C.-30° C. feeding temperature and 60%-70% air humidity level, and feeding was terminated at the end of the fifth instar of the larvae.

A group without any additives was used as the blank group (Group 1), groups with addition of sodium selenite were used as the positive groups (Groups 2, 4, and 6), and groups with addition of the selenium-rich bacteria solutions were used as the experimental groups (Groups 3, 5 and 7). Effects of the selenium-rich bacteria solution on breeding of the selenium-rich black soldier flies were determined according to the indicators, i.e. growth performance of the larvae (survival rate, larva weight), bioconversion efficiency, and selenium content in the larva. See Table 4. Test method for each indicator was the same as that in Example 2.

TABLE 4
Growth performance of selenium-rich black solider fly larvae of Example 4

| | Selenium | | Larva weight | | Selenium |
|---|---|---|---|---|---|
| | content added | Survival | (mg per | Bioconversion | content in the |
| Group | (mg/kg) | rate (%) | larva) | efficiency (%) | larva (μg/g) |
| 1 | 0.00 | 99.37 ± 1.16 | 215.78 ± 5.15 | 12.03 ± 1.64 | ND |
| 2 | 100.00 | 87.12 ± 6.53 | 183.72 ± 9.71 | 9.32 ± 0.27 | 96.00 ± 3.85 |
| 3 | 100.00 | 96.78 ± 1.67 | 236.02 ± 4.42 | 10.84 ± 0.14 | 146.91 ± 10.28 |
| 4 | 200.00 | 89.27 ± 3.40 | 156.56 ± 11.10 | 6.45 ± 0.44 | 262.67 ± 27.31 |
| 5 | 200.00 | 95.19 ± 0.73 | 202.66 ± 11.25 | 9.90 ± 0.59 | 391.12 ± 36.15 |
| 6 | 300.00 | 83.17 ± 0.97 | 122.15 ± 5.66 | 4.62 ± 0.35 | 461.95 ± 11.76 |
| 7 | 300.00 | 93.76 ± 1.67 | 151.02 ± 2.03 | 8.89 ± 0.49 | 673.10 ± 7.80 |

Note:
ND denotes a value below the detectable limit.

It can be concluded from Table 4 that sodium selenite exerts an inhibitory effect on the black soldier fly larvae. Specifically, the larvae survival rate, weight, and bioconversion efficiency drop with the increase of sodium selenite concentration. On the other hand, the selenium-rich bacteria solution (the experimental groups) can reduce the inhibitory effect, improve absorption of the larvae to selenium and increase the selenium content in the larva.

### Example 5

Step 1, Enterococcus faecalis derived from intestinal tract of the black soldier fly was activated, then inoculated to a MRS culture medium, and cultivated at 37° C. for 12 h to obtain a Enterococcus faecalis seed liquid with a bacteria content of 10⁸-10⁹ CFU/mL;
Bacillus subtilis derived from intestinal tract of the black soldier fly was activated, then inoculated to an LB culture medium, and cultivated at 37° C. for 12 h to obtain a Bacillus subtilis seed liquid with a bacteria content of 10⁸-10⁹ CFU/mL;
Step 2, the Enterococcus faecalis seed liquid was inoculated to a MRS culture medium at an inoculum size of 2%, and fermented for 6 h; then sodium selenite was added to the fermentation broth to enable the fermentation broth to have a selenium content of 450 μg/mL, and the fermentation broth was then cultivated at 37° C. for 36 h to obtain a selenium-rich bacteria solution of Enterococcus faecalis with a bacteria content of 10⁹ CFU/mL;
the Bacillus subtilis seed liquid was inoculated to an LB culture medium at an inoculum size of 2%, and fermented for 6 h; then sodium selenite was added to the fermentation broth to enable the fermentation broth to have a selenium content of 450 μg/mL, and the fermentation broth was then cultivated at 37° C. for 36 h to obtain a selenium-rich bacteria solution of Bacillus subtilis with a bacteria content of 10⁹ CFU/mL; and
Step 3, 6-day-old black soldier fly larvae were selected to be fed; the above two selenium-rich bacteria solutions were mixed according to a ratio of 1:1, and added to a feed to enable the feed to have a total selenium content of 100, 200, 300 mg/kg (wet weight), respectively; where the feed consisted of bean pulp, bran and grass meal at a ratio of 3:5:2, a moisture content of the feed was controlled to 75%; then the black soldier fly larvae were fed with the feed at a ratio of 150 g dry feed per 150 larvae (the ratio of dry feed to larva was 1.0) in conditions of 28° C.-30° C. feeding temperature and 60%-70% air humidity level, and feeding was terminated at the end of the fifth instar of the larvae.

A group without any additives was used as the blank group (Group 1), groups with addition of sodium selenite were used as the positive groups (Groups 2, 4, and 6), and groups with addition of the selenium-rich bacteria solutions were used as the experimental groups (Groups 3, 5 and 7). The effects of the selenium-rich bacteria solution on breeding of the selenium-rich black soldier flies were determined according to the indicators, i.e. growth performance of the larvae (survival rate, larva weight), bioconversion efficiency, and selenium content in the larva. See Table 5. Test method for each indicator was the same as that in Example 2.

TABLE 5
Growth performance of selenium-rich black solider fly larvae of Example 5

| | Selenium | | Larva weight | | Selenium |
|---|---|---|---|---|---|
| | content added | Survival | (mg per | Bioconversion | content in the |
| Group | (mg/kg) | rate (%) | larva) | efficiency (%) | larva (μg/g) |
| 1 | 0.00 | 97.32 ± 1.14 | 211.34 ± 5.05 | 11.79 ± 1.60 | ND |
| 2 | 100.00 | 85.33 ± 6.40 | 179.95 ± 9.51 | 9.12 ± 0.26 | 94.02 ± 3.78 |
| 3 | 100.00 | 95.63 ± 0.77 | 199.52 ± 3.73 | 9.16 ± 0.11 | 124.19 ± 8.69 |
| 4 | 200.00 | 87.43 ± 3.33 | 153.34 ± 10.87 | 6.32 ± 0.43 | 257.27 ± 26.74 |
| 5 | 200.00 | 94.00 ± 1.44 | 173.32 ± 9.51 | 8.37 ± 0.50 | 330.64 ± 30.56 |
| 6 | 300.00 | 81.46 ± 0.96 | 119.64 ± 5.54 | 4.53 ± 0.35 | 452.45 ± 11.52 |
| 7 | 300.00 | 93.92 ± 1.47 | 127.67 ± 1.71 | 7.51 ± 0.42 | 569.03 ± 6.60 |

Note:
ND denotes a value below the detectable limit.

It can be concluded from Table 5 that sodium selenite exerts an inhibitory effect on the black soldier fly larvae. Specifically, the larvae survival rate, weight, and bioconversion efficiency drop with the increase of sodium selenite concentration. On the other hand, the selenium-rich bacteria solution (the experimental groups) can reduce the inhibitory effect, and improve absorption of the larvae to selenium and increase the selenium content in the larva.

### Example 6

The selenium-rich black soldier fly larvae (selenium content was 582.22 μg/g _{dry weight}) fed and obtained in Example 3 were subjected to starvation treatment for 1 day, and dried with a microwave oven till the moisture content was less than 8% to obtain dry larvae.

### Example 7

The selenium-rich black soldier fly larvae (selenium content was 582.22 μg/g _{dry weight}) fed and obtained in Example 3 were subjected to starvation treatment for 1 day, then dried with hot wind and smashed. Petroleum ether was added to the smashed larvae at a solid-liquid ratio of 1:60 -1:100 g/mL, and was then subjected to processing at 100° C. for 3 h to extract larval oil and to obtain defatted selenium-rich black soldier fly larvae protein powder. The protein powder was detected to have a selenium content of 973.65 μg/g.

### Example 8

240 Hy-Line brown laying hens were selected and divided into 4 groups, each group had 4 duplicates and each duplicate had 15 hens. Basic rations were prepared by referring to the US NRC standard. The selenium-rich black soldier fly larvae fed and obtained in Example 3 were subjected to starvation treatment for 1 day, and subsequently to freezing treatment. These selenium-rich frozen fresh larvae were added to a feed for the laying hens in an additive amount of 10%. Effect of the selenium-rich black soldier fly larvae on the breeding of laying hens was observed.

TABLE 6
Grouping of experiments of laying hens

| | Additive amount of black | Selenium content of the |
|---|---|---|
| Group | soldier fly larvae (%) | feed for laying hens (μg/g) |
| A | 0 | 0.4 |
| B | 10 | 0.4 |
| C | 10 | 0.7 |
| D | 10 | 1.3 |

The preliminary trial period lasted for 1 week and the trial period lasted for 4 weeks. During the trials, the hens were kept in a 3-storey stepped cage in a closed henhouse, with 3 hens in each cage. Breeding and management were carried out according to conventional methods in a poultry farm. A henhouse illumination automatic control system with artificial lighting was used, and was set to 16 h of lighting/8 h of darkness, with 20 1× illumination intensity 25±5° C. room temperature, and 40%-70% relative humidity. Ad libitum access to food and water was provided. Sterilization was carried out once a week, and the hens for trial were subjected to routine vaccination.

Effects of selenium-rich black soldier fly larvae on productive performance and egg quality of laying hens are shown in Table 7.

TABLE 7
Effects of selenium-rich black soldier fly larvae on
productive performance and egg quality of laying hens

| | A | B | C | D |
|---|---|---|---|---|
| Laying rate (%) | 90.97 ± 8.19 | 92.36 ± 2.15 | 92.02 ± 1.57 | 92.36 ± 1.07 |
| Egg weight (g) | 64.17 ± 1.00 | 66.50 ± 0.86 | 67.20 ± 1.01 | 67.03 ± 0.96 |
| Daily feed intake | 113.64 ± 0.55 | 113.80 ± 0.30 | 113.95 ± 0.31 | 114.00 ± 0.56 |
| (g/day/hen) | | | | |
| Albumen height (mm) | 7.05 ± 1.60 | 7.22 ± 0.80 | 7.29 ± 1.19 | 7.10 ± 0.71 |
| Haugh unit | 81.06 ± 11.54 | 82.05 ± 4.35 | 82.39 ± 7.25 | 81.56 ± 4.19 |
| Eggshell | 0.39 ± 0.02 | 0.39 ± 0.03 | 0.40 ± 0.02 | 0.40 ± 0.03 |
| thickness (mm) | | | | |
| Eggshell | 49.12 ± 6.56 | 52.90 ± 5.09 | 52.14 ± 9.43 | 52.76 ± 8.43 |
| strength (kg/m²) | | | | |
| Egg shape index | 1.30 ± 0.05 | 1.30 ± 0.04 | 1.31 ± 0.03 | 1.31 ± 0.03 |

Effect of selenium-rich black soldier fly larvae on selenium content in egg is shown in Table 8.

TABLE 8
Effect of selenium-rich black soldier fly larvae on selenium content in egg

| | A | B | C | D |
|---|---|---|---|---|
| Selenium | 41.10 ± 9.52^{c} | 42.35 ± 7.88^{c} | 88.72 ± 8.84^{b} | 179.68 ± 25.40^{a} |
| content in egg | | | | |
| (μg/100 g) | | | | |

It can be concluded from the above results that the addition of selenium-rich black soldier fly larvae is conducive to enhancing the egg laying capacity of laying hens and the increase of laying rate and egg weight. Moreover, no harmful effect is made to the various indicators of egg quality. With the increase of selenium content in the feed for laying hens, the content of the selenium element accumulated in the eggs increased as well.

The above examples only demonstrate a few of the many implementations of the present invention. Although the description is relatively specific and detailed, it should not be understood as limiting the patent scope of the present invention. It is crucial to acknowledge that for those skilled in the field, several modifications and improvements can be made under the circumstance that the modifications do not depart from the inventive concept, which are within the scope of protection of the present invention.

What is claimed is:

1. A method for feeding black soldier fly larvae with a selenium-rich bacteria solution, comprising the following steps:
step 1, inoculating an intestinal bacterial strain of black soldier fly to a culture medium after activating the intestinal bacterial strain of black soldier fly, cultivating at 30° C.-37° C. for 12-18 h to reach a bacteria content of 10⁸-10⁹ CFU/mL so as to serve as an intestinal bacteria seed liquid of black soldier fly; then inoculating the intestinal bacteria seed liquid of black soldier fly to a fermentation medium at an inoculum size of 1%-3%, and cultivating at 30° C.-37° C. for 6-12 h till the log phase, so that a fermentation broth of log phase has a bacteria content of 10⁸-10⁹ CFU/mL;
step 2, adding sodium selenite to the fermentation broth of log phase to enable the fermentation broth to have a sodium selenite concentration of 10-600 μg/mL, then cultivating the fermentation broth at 30° C.-37° C. for 24-36 h to obtain a selenium-rich bacteria solution; and
step 3, selecting 4-to-6-day-old black soldier fly larvae to be fed, adding the selenium-rich bacteria solution to a feed to enable the feed to have a selenium content of 5-500 mg/kg, controlling a moisture content of the feed to 60%-75%, feeding the black soldier fly larvae with the feed at a dry feed to larva ratio of 0.5-1.0 g per larva in conditions of 28° C.-30° C. feeding temperature and 60%-70% air humidity level, and terminating feeding at the end of the fifth instar of the larvae.

2. The method for feeding black soldier fly larvae with the selenium-rich bacteria solution according to claim 1, wherein the selenium-rich bacteria solution in step 2 has a bacteria content of 10⁹-10¹⁰ CFU/mL.

3. The method for feeding black soldier fly larvae with the selenium-rich bacteria solution according to claim 1, wherein the selenium-rich bacteria solution in step 2 has a nano-selenium content of 5-350 μg/mL.

4. The method for feeding black soldier fly larvae with the selenium-rich bacteria solution according to claim 1, wherein the intestinal bacterial strain of black soldier fly is selected from a group consisting of Lactococcus lactis, Lactobacillus acidophilus, Lactobacillus plantarum and Enterococcus faecalis, and complex microbial inoculants thereof with Bacillus subtilis.

5. The method for feeding black soldier fly larvae with the selenium-rich bacteria solution according to claim 1, wherein the feed in step 3 is chicken feed or plant-based feed, with the plant-based feed being prepared by bean pulp, bran, and grass meal according to a weight ratio of 3:5:2.
